(19) United States
(12) Patent Application Publication
(10) Pub. No.: US 2026/0192011 A1
(43) Pub. Date: Jul. 9, 2026

(54) ARRANGEMENT AND METHOD FOR PREVENTING THE SPREAD OF MICROBES

(71) Applicant: ELVEION OY, Helsinki (FI)

(72) Inventor: Veikko ILMASTI, Helsinki (FI)

(73) Assignee: ELVEION OY, Helsinki (FI)

(21) Appl. No.: 18/865,522

(22) PCT Filed: Sep. 13, 2023
(86) PCT No.: PCT/FI2023/050520

§ 371 Date: Nov. 13, 2024

(51) Int. Cl. A61L 9/22

(52) U.S. Cl. CPC A61L 9/22

(57) ABSTRACT

An arrangement (1) and a method for preventing the spread of microbes to a person (2) and/or from the person (2) in an indoor environment (3). The arrangement (1) comprises at least one ion-producing element (4) with a tip (5) that emits negative ions and is connectable to a power source (6) and is configured to emit a conical ion beam (7) containing negative ions as a negative direct voltage is supplied to said ion-emitting tip (5) from the power source (6), said tip (5) emitting negative ions being installable in the indoor environment (3), such as above the person (2). The orientation of the tip (5) emitting negative ions is adjustable in order to direct the ion beam (7) towards the person (2) in the indoor environment (3).

### FIELD OF THE INVENTION

The invention relates to an arrangement and a method for preventing the spread of microbes from a person and/or to the person in an indoor environment.

### BACKGROUND OF THE INVENTION

Air purification solutions based on air ionization are commonly known. In these solutions, negatively charged ions purify the air by separating, inter alia, microbes, such as bacteria and viruses. However, such air purifiers are not engineered to pre-vent microbes from being released from a contamination source, such as a person, into the air of an indoor environment and/or from spreading to other persons in the same indoor environment.

### OBJECTIVE OF THE INVENTION

The objective of the invention is to provide an arrangement and a method allowing the above-mentioned drawbacks to be diminished.

### SUMMARY OF THE INVENTION

The objective of the invention can be achieved by means of an arrangement as defined in claim 1 and a method as defined in claim 13.

The arrangement according to the invention comprises at least one ion-producing element having an ion-emitting tip connectable to a power source and configured to emit a conical ion beam as the power source supplies a negative high direct voltage to the said tip emitting negative ions. The tip emitting negative ions can be installed in an indoor environment. The orientation of the tip emitting negative ions is adjustable in order to direct the ion beam, such as center axis of the ion beam, towards a person or group of persons in the indoor environment.

In the method according to the invention, at least one ion-producing element having a tip emitting negative ions is placed in an indoor environment, a negative high direct voltage is supplied to said tip emitting negative ions, a conical ion beam containing negative ions is emitted from the tip emitting negative ions, and the orientation of the tip emitting negative ions is adjusted in order to direct the ion beam, such as center axis of the ion beam, towards a person or group of persons in the indoor environment.

The invention may provide significant advantages. In the arrangement according to the invention, the orientation of the tip emitting negative ions is adjusted in order to direct the ion beam towards a person in the indoor environment. This allows the number of microbes spreading from the person to the indoor environment to be reduced and/or the microbes present in the indoor air from coming into contact with the person needing protection.

The protective effect of the arrangement and method according to the invention is achieved in three different ways:

As the ion beam produced by the ion-emitting tip is directed towards the person or group of persons in need of protection, the ion beam imparts a negative electric charge to the person. Ions are separated from the outer edge of the ion beam into the indoor air. The negative ions dispersed into the indoor environment from the ion beam impart a negative electric charge to the air and to the microbes present in the indoor environment. The electric charge that the ion beam directs towards the person or group of persons and causes to the person is higher than the one that the negative ions dispersed into the indoor air cause to the microbes. The person having a negative electric charge and the negatively charged microbes repel each other. As the negatively charged microbes outside the ion beam travel to the outer edge of the ion beam, the ion beam directs a repelling force towards the microbes, thus preventing the microbes from coming into contact with the person or group of persons needing protection.

As the ion beam of the ion-emitting tip is directed towards the person or group of persons emitting microbes, such as an infected person secreting viruses or bacteria, the ion beam imparts a negative electric charge to both the person and the microbes emitted by the person. Besides, ions are released from the outer edge of the ion beam into the indoor air. The negative ions dispersed into the indoor environment give the indoor air a negative electric charge that is lower than the negative electric charge received by the person or group persons towards which the ion beam was directed. This creates a condition where no negatively charged microbes spread from the microbe-emitting person to the negatively charged indoor air.

The orientation of the ion-producing tip is adjustable in order to allow the ion beam to be accurately directed towards the person or group of persons emitting microbes and/or needing protection.

Further, the negative ions dispersed into the indoor environment from the ion beam deliver a negative electric charge to the indoor air, the microbes in the indoor air and the untargeted persons in the indoor environment. Because the negative charges repel each other, the negatively charged microbes do not come into contact with the negatively charged person. The negative electric charge caused to the person by the ions dispersed into the indoor air is lower than in a situation where the ion beam is directed towards the person or group of persons.

The arrangement and method according to the invention are especially applicable to the interiors of different types of vehicles, such as buses, trains, airplanes and ships. Tips emitting negative ions are installed in the interior of the vehicle, such as above the passenger seats, and are oriented towards the persons sitting on the passenger seats.

The arrangement and method according to the invention are also applicable to the interiors of buildings, such as the interiors of hospitals, shopping centers and sports halls. In a hospital, for example, the arrangement can be installed at the top of a patient room, while the ion-emitting tip is oriented towards a patient/an unwell person lying on a bed, by directing the ion beam, such as the center axis of the ion beam, emitted by the tip, towards the patient. If the bed is moved in the room, the orientation of the ion-emitting tip is adjusted so that the ion beam is directed to-wards the patient. The ion beam directed towards the patient prevents the microbes spreading from the patient to the air in the room.

The arrangement and method according to the invention are also useful for preventing the spread of odors.

### BRIEF DESCRIPTION OF FIGURES

In the following, the invention will be explained in more detail by means of examples, with reference to the accompanying drawings where

FIG. 1 is a view of an interior of a structure, such as a building or vehicle, comprising an arrangement according to an embodiment of the invention,

FIGS. 2 and 3 are views of the arrangement shown in FIG. 1,

FIG. 4a is a view of an interior of a building, such as a hospital patient room, lacking an arrangement according to invention,

FIG. 4b is a view of the interior shown in FIG. 4a, having an arrangement according to an embodiment of the invention installed therein,

FIG. 5 is a view of a public facility, such as a recreation facility, having an arrangement according to an embodiment of the invention installed therein,

FIG. 6a is a side view of an interior of a vehicle, such as an airplane or train, having an arrangement according to an embodiment of the invention installed therein, and

FIG. 6b is a front view of the interior shown in FIG. 6a.

### DETAILED DESCRIPTION

The figures show an arrangement 1 for preventing the spread of microbes, such as viruses (COVID-19, for example), bacteria, spores and other microorganisms, from a person 2 or group of persons to the air of an interior 3 and/or from the air of the interior 3 air to the person 2 or group of persons. The arrangement 1 prevents the spread of microbes from the person 2 or group of persons to the air of the interior 3 and/or from the air of interior 3 to the person 2 or group of persons. The interior 3 belongs to a structure, such as a hospital, terminal, school, shopping center, sports hall, station etc., or, to a vehicle, such as an airplane, bus, train or ship.

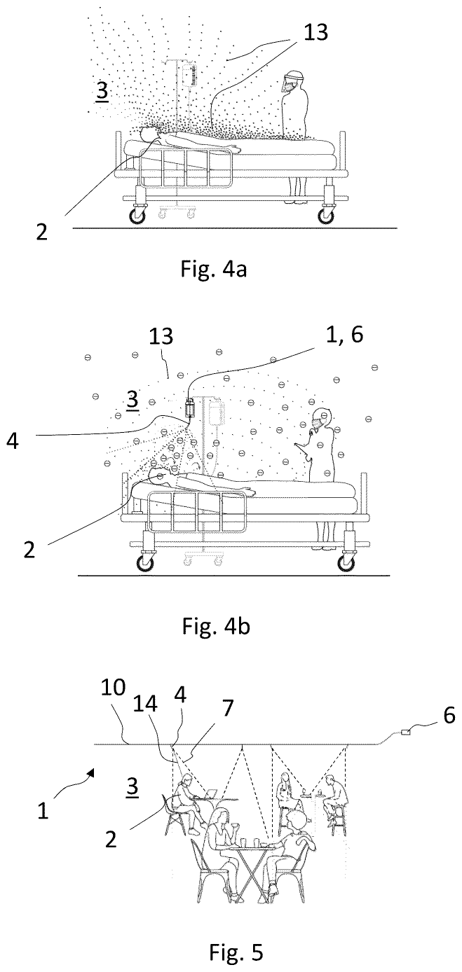

The arrangement 1 comprises one or more ion-producing elements 4 having a tip 5 emitting negative ions. Typically, each ion-producing element 4 comprises a single ion-emitting tip 5. The ion-emitting tip 5 is positioned at an end of the ion-producing element 4. Further, the arrangement may comprise a power source 6 to which the ion-producing element 4 can be connected. The power source 6 is arranged to supply a negative direct voltage of 4.5 kV to 5.5 kV, typically 4.9 to 5.1 kV, such as 5 kV, to the ion-producing element 4 and the ion-emitting tip 5. The power source 6 is arranged to supply a direct current of 0.4 mA- 1.6 mA, typically 0.8 mA- 1.2 mA, such as 1 mA, to the ion-producing element 4 and the ion-emitting tip 5. The electric current supplied to the ion-emitting tip 5 is low enough to not pose a danger to a human body. The power source 6 can be connected to a grid supplying an alternating current, such as 220 V 50/60 Hz or 110 V, to the power source 6. The power source 6 transforms the grid voltage into a negative direct voltage of a desired magnitude. As an alternative or addition to the grid supply, the power source 6 can be equipped with a battery or accumulator whose voltage is transformed into a negative direct voltage of a desired magnitude. The arrangement 1 can be portable, as shown in the embodiment according to FIG. 4b.

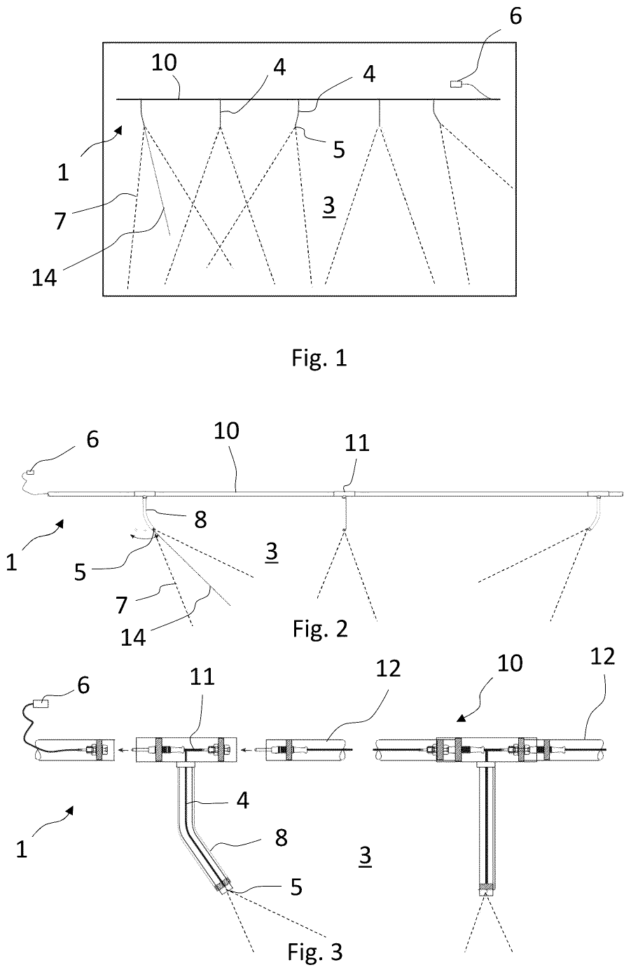

The ion-emitting tip 5 is configured to emit a conical ion beam 7 containing negative ions when a negative direct voltage (high voltage) is supplied to said ion-emitting tip 5. The ion beam 7 has the shape of a spherical cone. The ion-producing element 4 can be installed in the interior 3. The ion-producing element 4 is installed at the top of the interior 3, such as on the ceiling of the interior, or above a passenger seat of a vehicle, or, at the top of a passageway or some other interior 3 of the vehicle. Typically, as shown in FIGS. 1 to 3 and 5 to 6, the arrangement 1 comprises several ion-producing elements 4, each of them having an ion-emitting tip 5. Each ion-producing element 4 may comprise a single ion-producing tip 5. In the embodiment shown in FIG. 4b, the arrangement 1 is enclosed in a housing to make the arrangement 1 portable.

If the arrangement 1 comprises several ion-emitting tips 5, the adjacent ion-emitting tips 5 are spaced-apart. The distance between the adjacent ion-emitting tips 5 is at least 50 cm, typically at least 40 cm, if the arrangement installed in an interior 3 of a vehicle. Typically, at least one ion-emitting tip 5 is provided above each passenger seat, or, above and between two adjacent passenger seats.

When the arrangement 1 is installed in an interior 3 of a building, such as at the top of a room of on the ceiling thereof, the distance between the adjacent ion-emitting tips 5 can be 4 to 8 meters. The distance between the adjacent ion-emitting tips 5 and their orientation are chosen such that the ion beams 7 emitted by the tips 5 do not interfere with each other, for instance, the ion beams 7 do not intersect or overlap each other. The ion-emitting tips 5 are positioned at a height of 2.5 to 3 m from the floor of the interior 3. A distance of 0.5 to 1.5 meters is provided between a person and the tip 5.

If the arrangement 1 comprises several ion-producing elements 4, the arrangement 1 may comprise a main supply line 10 from which ion-producing elements 4 branch out, as more specifically shown in FIGS. 1 to 3. The main supply line 10 is enveloped by an electrical insulation. The arrangement 1 may also comprise at least one power source 6 arranged to supply a negative direct voltage to the ion-producing elements 4 and the ion-emitting tips 5. The power source 6 can be connected to the main supply line 10.

The ion-producing element 4 and/or the ion-emitting tips 5 can be made from a conductive wire, such as stainless steel wire. The diameter of the ion-producing element 4 and/or the ion-emitting tip 5 is 0.1 to 0.25 mm, typically 0.15 to 0.21, such as 0.18 mm. The diameters of the ion-producing element 4 and of the ion-emitting tips 5 can be equal in size. The diameters of the ion-producing element 4 and of the ion-emitting tips 5 can be constant over the entire length thereof. The ion-emitting tip 5 has a flat end. The length of the ion-emitting tip 5 is at least 10 cm, typically 15 to 20 cm.

Once the arrangement 1 has been installed in the interior 3, the orientation of the ion-emitting tip 5 can be adjusted in order to direct the ion beam 7, such as the center axis 14 of the ion beam 7, towards a person 2 or group of persons. The ion-producing element 4 and/or the ion-emitting tip 5 can be directed towards a person 2 or group of persons. The ion-producing element 4 can be turnable and/or bendable for the adjustment of the orientation of the ion-emitting tip 5. If the arrangement 1 comprises several ion-emitting tips 5, the orientation of each ion-emitting tip 5 can be adjusted independently.

The ion-producing element 4 can be enclosed in a protective casing 8, such as a protective sleeve or protective tube. The protective casing 8, such as the protective tube, can be turnable or bendable. The ion-emitting tip 5 protrudes out of the protective tube 8, or, the tip 5 is flush with the outer surface of the protective tube 8. The protective tube 8 is made of a non-conductive material, such as plastic. The length of the protective tube 8, and/or of the protective tube 8 and the ion-producing element 4, is at least 10 cm, typically 15 to 20 cm. This allows the protective tube 8 to be manually bent and/or turned without difficulty, for the purpose of adjusting the orientation of the ion-emitting tip 5.

As shown in FIGS. 2 and 3, the main supply line 10 may comprise branch sections 11 from which the ion-producing elements 4 branch out, as well as base sections 12 between the branch sections 11. The branch sections 11 and/or the ion-producing elements 4 can be turnable and/or bendable with respect to the base sections 12 to allow the orientation of the ion-emitting tips 5 to be adjusted. The base sections 12 are fixedly installed in the interior 3. The base sections 12 are enclosed in protective casings, such as protective tubes. The branch sections 11 are enclosed in protective casings, such as protective tubes. The ion-producing element 4 and the protective casing 8 of the ion-producing element 4 can be bendable. The ion-producing element 4 is connected to the branch section 11.

The base sections 12 and the branch sections 11 of the main supply line 10 are connected to each other in such a way that the branch sections 11 are pivotable and/or turnable with respect to the base sections 12. The branch sections 11 and the base sections 12 can be provided with pivoting joints to allow the branch sections 12 to be twisted/turned with respect to the base sections 12. As shown in FIG. 3, the end of the branch 12 can comprise a pin, and the end of the base section 11 can comprise a seat adapted to accommodate the pin, or vice versa.

The orientation of the ion-emitting tip 5 is adjusted manually, or, the arrangement 1 may include a mechanism configured to orient the ion-emitting tip 5. The mechanism can be a turning device connected to the ion-producing element 4 or the protective casing 8. The mechanism can be remotely controlled.

FIG. 4b shows a portable arrangement 1 where the power source 6 comprises a battery, such as a 9 V battery, or a rechargeable battery, a transformer for transforming the voltage into a direct voltage of a desired magnitude. The battery/accumulator and the transformer are enclosed in a casing. The arrangement 1 may comprise a single ion-producing element 4 and a single ion-producing tip 5. The ion-producing element 4 is connected to the power source 6. The ion-producing element 4 protrudes from the casing of the power source 6. As an alternative or addition to the battery/accumulator, the power source 6 can be connectable to an electric grid by an electric wire.

The arrangement is operated as follows. A negative direct voltage (high voltage) is supplied to each ion-producing element 4 and ion-emitting tip 5. The negative direct voltage is 4.5 kV to 5.5 kV, typically 4.9 to 5.1 kV, such as 5 kV, in magnitude. Besides, a direct current is supplied from the power source 6 to each ion-emitting tip 5. The current is 0.4 mA to 1.6 mA, typically 0.8 mA- 1.2 mA, such as 1 mA, in magnitude. A conical ion beam 7 containing negative ions leaves the ion-emitting tip 5. The ion beam 7 has the shape of a spherical cone. The speed of the ions of the ion beam is 8 to 12 m/s, typically 10 m/s. Typically, the ions of the ion beam travel a distance of three meters, after which the ions slow down and the ions disperse into the indoor environment 3. Ions are also separated from the outer edge of the ion beam 7 into the interior 3. The air of the interior 3 becomes negatively charged.

The ion yield from the tip emitting negative ions is 4×10¹¹ to 6×10¹¹ ions per second, typically 4.5×10¹¹ to 5.5×10¹¹ ions per second. The number of the ion-emitting tips 5 and/or the operational parameters of the arrangement 1 are chosen to give an average number of negative ions of 2000 to 10 000 cm³ in the interior 3. A person 2 or group of persons, toward which the ion beam 7 is directed, gain a negative electric charge. The operational parameters of the arrangement 1 are chosen to provide a number of negative ions of 20000 to 500000ions/cm³, such as 50000 to 120000ions/cm³, in proximity to the person 2, such as within 0.5 meters from the person, toward whom the ion beam 7 is directed. Besides, the microbes present in the interior 3, such as the microbes emitted by the person 3, also become negatively charged.

The orientation of the ion-emitting tip 5 installed in the interior 3 is adjusted in order to direct the ion beam 7, such as the center axis 14 of the ion beam. The orientation of the ion-emitting tip 5 is adjusted after installing the arrangement 1 in the interior 3. If the arrangement 1 comprises several ion-emitting tips 5, the orientation of each tip 5 is adjusted independently. The ion beam 7, such as the center axis of the ion beam 7, is directed towards the person 2 or group of persons which, for example, is a person 2 needing protection and/or an infected person 2 emitting microbes. The orientation of the ion-emitting tip 5 is adjusted by turning and/or bending the ion-producing element 4 in order to direct the ion-emitting tip 5 towards the person or group of persons. The orientation of the ion-emitting tip 5 is adjusted manually, or, the arrangement 1 may include a mechanism configured to orient the ion-emitting tip 5. The mechanism can be a turning device connected to the ion-producing element 4 or the protective casing 8. The mechanism can be remotely controlled. After adjusting the orientation of the ion-emitting tip 5, tip 5 is adapted to keep its adjusted orientation independently. Thus, the orientation of the tip 5 remains adjusted, for example, without holding hands.

If the ion beam 7, such as the center axis 14 of the ion beam, is directed to a person 2 or group of persons, such as an infected/unwell person 2 lying on a bed, the ion beam 7 imparts a negative electric charge to both the person 2 and the microbes emitted by the person. The negative ions dispersed from the ion beam 7 into the interior 3 impart a negative electric charge to the air of the interior 3. This creates a condition where no negatively charged microbes disperse from the microbe-emitting person 2 or group of persons into the negatively charged air of the interior 3. This is illustrated in FIGS. 4a and 4b of which FIG. 4a lacks an arrangement 1 according to the invention in the interior. In FIG. 4b, the interior 3 contains an arrangement 1 according to an embodiment of the invention whose ion-producing tip 5 is directed towards the person 2 emitting microbes 13. As can be seen, the number of microbes 13 dispersing from the person 2 into the interior 3 is significantly lower in FIG. 4b where the ion beam 7 is directed towards the person 2.

FIG. 4b shows an interior 3 of a building, such as a patient room of a hospital, provided with a portable, battery-powered or accumulator-powered arrangement 1 according to an embodiment of the invention. The arrangement 1 includes a power source 6 comprising a battery, such as a 9 V battery, or a rechargeable accumulator. The power source 6 also comprises a transformer for transforming the voltage of the battery or accumulator into a negative high-voltage of a desired magnitude. The arrangement 1 also comprises an ion-producing element 4 having an ion-emitting tip 5 at its end. The arrangement may comprise a single ion-producing element 4 and a single ion-emitting tip 5. The power source 6 also comprises a casing enclosing the battery/accumulator and transformer. The ion-producing element 4 is connected to the power source 6. The ion-producing element 4 protrudes from the casing of the power source 6. The casing may comprise a fastening member, such as a hook or loop, to allow the arrangement 1 to be fastened/hung up. In FIG. 4b, the arrangement 1 is hanging on a medical dropper. Once the arrangement 1 is in place, the orientation of the ion-emitting tip 5 is adjusted in order to direct the ion beam 7, such as center axis 14 of the ion beam 7, towards the person 2. The orientation of the ion-emitting tip 5 is adjusted by turning and/or bending the ion-producing element 4. As an alternative or addition to the battery/accumulator power, the arrangement 1 can be connectable to a grid.

FIG. 5 is a view of an arrangement 1 comprising several ion-producing elements 4, such as the arrangement shown in FIGS. 2 and 3. The arrangement 1 is installed at the top of an interior 3 of a building, such as on the ceiling of a restaurant hall. The ion beams 7 emitted by the ion-emitting tips 5, such as the center axes 14 of the ion beams 7, are directed towards the tables and/or persons 2 sitting at the tables. The ion beams 7 are directed by adjusting the orientation of the ion-emitting tips 5. The ion beam 7 emitted by the tip imparts a negative electric charge to the person 2 sitting at the table and the microbes emitted by this person 2. This allows the spread of the microbes to the air of the interior 3 to be reduced. Besides, the ions of the ion beam 7 are dispersed into the air of the interior 3, giving the air a negative electric charge.

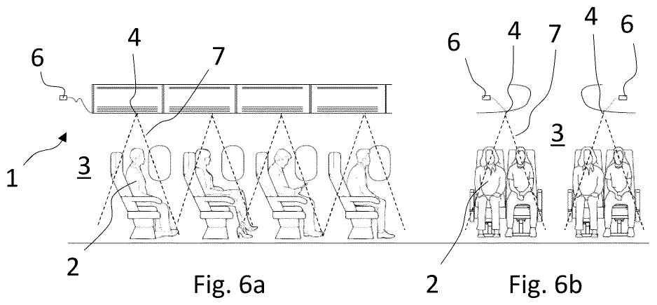

FIGS. 6a and 6b show an interior 3 of a vehicle, such as an aircraft or train cabin having several passenger seats. An arrangement 1 according to the invention, which can be arrangement 1 shown in FIGS. 2 and 3, is installed above the passenger seats, at the top of the interior 3, such as on the ceiling or on an overhead rack above the passenger seats. An ion-emitting tip 5 is provided above each passenger seat or between each two adjacent passenger seats, above the seats. The orientation of the tip 5 is adjusted in order to direct the ion beam 7, such as the center axis 14 of the ion beam 7, towards a person 2 sitting on one of the passenger seats. The ion beam 7 emitted by the tip 5 imparts a negative charge to the person 2 sitting on the passenger seat and to the microbes emitted by this person, thus reducing the spread of the microbes to the air of the interior 3. The ions of the ion beam 7 disperse into the air of the interior 3 and give air of the interior 3 a negative electric charge. Any other persons present in the interior 3 are also given a negative electric charge by the ion beam 7 and/or the negative ions dispersed into the indoor air 3, resulting in that the negatively charged microbes and negatively charged persons repel each other. Thus, the negatively charged persons are not infected by the microbes.

If the arrangement 1 is installed in an interior 3 of a vehicle, the ion-emitting tips 5 can be fixedly installed and unadjustable in orientation. In this case, the orientation of the tips 5 is adjusted in a desired manner, such as towards the passenger seats while installing the arrangement 1.

In all the above-described embodiments, the orientation of the ion-emitting tip 5 is intentionally adjusted so that the ion beam 7 emitted by the tip 5, such as the center axis 14 of the ion beam 7, is directed towards a person 2 or group of persons present in an indoor environment 3, such as a person emitting microbes and/or a person needing protection. The person 2 or group of persons gain a negative electric charge. If the person is infected, the microbes, such as viruses and bacteria, emitted by this person also gain a negative electric charge. The negative ions dispersing into the indoor air 3 from the ion beam 7 impart a negative electric charge to the indoor air 3. This allows the spread of the microbes from the person 2 into the indoor air to be reduced.

Correspondingly, the ion beam 7 directed towards the person or group of persons needing protection in the indoor environment 3 imparts a negative electric charge to the person or group of persons. The electric charge from the ion beam directed towards to the person 2 and caused to this person is higher than the one that the negative ions dispersed from the ion beam 7 into the indoor air 3 cause to the microbes. The person having a negative electric charge and the negatively charged microbes repel each other. As the negatively charged microbes outside the ion beam 7 travel to the outer edge of the ion beam 7, the ion beam 7 directs a repelling force towards the microbes, thus preventing the microbes from coming into contact with the person 2 or group of persons to be protected.

The ion beam 7, such as the center axis 14 of the ion beam 7, is directed towards the person 2 or group of persons to give the person 2 or group an adequately high negative electric charge.

It will be understood by a person skilled in the art that, with technology developments, the inventive basic idea can be implemented in various ways. Thus, the invention and its embodiments are not restricted to the above-described examples but may vary with the scope of the claims.

1. An arrangement for preventing the spread of microbes to a person or group of persons and the spread of microbes from the person or group of persons in an indoor environment, the arrangement comprising at least one ion-producing element equipped with a tip that emits negative ions and is connectable to a power source and is configured to emit a conical ion beam containing negative ions as a negative direct voltage is supplied to said ion-emitting tip from the power source, said tip emitting negative ions being installable in the indoor environment, such as above the person or group of persons, characterized in that the orientation of the tip emitting negative ions is adjustable in order to direct the ion beam, such as the center axis of the ion beam, towards the person or group of persons in the indoor environment

2. An arrangement as defined in claim 1, characterized in that the arrangement comprises several ion-producing elements whose tips emitting negative ions are spaced-apart, the distance preferably being at least 10 cm, such as at least 30 cm, typically at least 50 cm, the orientation of each tip emitting negative ions being independently adjustable.

3. An arrangement as defined in claim 1 or 2, characterized in that the ion-producing element is turnable and/or bendable for the adjustment of the orientation of the tip emitting negative ions.

4. An arrangement as defined in claim 1, characterized in that the ion-producing element is enclosed in a protective casing, such as a protective sleeve or protective tube, the protective casing being turnable and/or bendable.

5. An arrangement as defined in claim 1, characterized in that the diameter of the ion-producing element and/or the tip emitting negative ions is 0.1 to 0.25 mm.

6. An arrangement defined in claim 1, characterized in that the power source is configured to supply a negative direct voltage of between about 4.5 kV to 5.5 kV to the tip emitting negative ions, and/or the power source is configured to supply a negative direct current between about of 0.4 mA to 1.6 mA to the tip emitting negative ions.

7. An arrangement as defined in claim 1 characterized in that each ion-producing element comprises a single tip emitting negative ions.

8. An arrangement as defined in claim 1 characterized in that the arrangement is portable, and the arrangement comprises a power source enclosed in a casing and having a battery or a rechargeable accumulator, and/or is connectable to a grid, and the ion-producing element protrudes from the casing.

9. An arrangement as defined in claim 2, characterized in that the arrangement comprises a main supply line from which the ion-producing elements branch out, the ion-producing elements being turnable and/or bendable with respect to the main supply line.

10. A structure, such as a building or vehicle, having an interior, characterized in that the interior comprises an arrangement as defined in claim 1.

11. A structure as defined in claim 10, characterized in that the structure is a vehicle having several passenger seats in its interior, and the arrangement comprises several ion-producing elements whose tips emitting negative ions are provided above the passenger seats.

12. A structure as defined in claim 10, characterized in that the structure is a building, and the arrangement comprises several ion-producing elements whose tips emitting negative ions are spaced-apart at the top of the interior.

13. A method of preventing the spread of microbes from a person/group of persons and/or to the person/group of persons in an indoor environment, such as an interior of a building or vehicle, the method comprising: providing at least one ion-producing element having a tip emitting negative ions in the in the interior, supplying a negative direct voltage to said tip emitting negative ions, emitting a conical ion beam containing negative ions from the tip emitting negative ions, characterized in that the orientation of the tip emitting negative ions is adjustable in order to direct the ion beam, such as the center axis of the ion beam, towards the person or group of persons in the indoor environment, and the orientation of the tip emitting negative ions is adjusted in order to direct the ion beam, such as the center axis of the ion beam towards the person or group of persons.

14. A method as defined in claim 13, characterized in that the orientation of the tip emitting negative ions is adjusted by turning and/or bending the ion-producing element

15. A method as defined in claim 13, characterized in that several ion-producing elements are provided in the indoor environment, each of them having a tip emitting negative ions, and the orientation of each tip emitting negative ions is adjusted independently.
